(19) United States
(12) Patent Application Publication
(10) Pub. No.: US 2002/0055493 A1
(43) Pub. Date: May 9, 2002

(54) Phytosterol and/or phytostanol derivatives

(75) Inventors: David Carl Burdick, Binningen (CH); Gerard Moine, Riedisheim (FR); Daniel Raederstorff, Brunstatt (FR); Peter Weber, Malsburg-Marzell (DE)

Correspondence Address: Stephen M. Haracz, Esq., BRYAN CAVE LLP, 245 Park Avenue, New York, NY, 10167-0034, US

(73) Assignee: ROCHE VITAMINS INC.

(21) Appl. No.: 09/989,554

(22) Filed: Nov. 20, 2001

Related U.S. Application Data
(62) Division of application No. 09/448,356, filed on Nov. 23, 1999.

(30) Foreign Application Priority Data
Nov. 26, 1998 (EP) 98122412.4
Sep. 29, 1999 (EP) 99119337.6

(51) Int. Cl. A61K 31/56

(52) U.S. Cl. 514/169

(57) ABSTRACT

The present invention relates to a phytosterol and/or a phytostanol ester compound produced from the reaction of a phytosterol and/or a phytostanol with a polyunsaturated fatty acid (PUFA), wherein the polyunsaturated fatty acid has from 18 to 22 carbon atoms and at least three carbon-carbon double bonds. Processes for producing and compositions and a process for using such compositions are also provided.

### FIELD OF THE INVENTION

The present invention relates to polyunsaturated fatty acid esters of phytosterols and/or phytostanols and methods of making and using such compositions.

### BACKGROUND OF THE INVENTION

Phytosterols are plant sterols found, for example, in small amounts in vegetable oils such as corn, bean, or other plant oils, where they occur as free sterols, fatty acid esters, and glycosides. Phytosterols are structurally similar to cholesterol, the main differences occurring in the carbon skeleton of their side chains. A number of different phytosterol structures are found in nature. The most common of these structures are campesterol, beta-sitosterol, and stigmasterol. Reduction of phytosterols yields saturated phytosterols, called phytostanols, such as campestanol or sitostanol, which also occur naturally in small amounts. A normal human diet typically leads to ingestion of less than one half gram a day of such substances in various forms.

It is known that ingestion of phytosterols and/or phytostanols in defined amounts (e.g., several grams a day or more) may reduce blood serum cholesterol levels. It is assumed that free phytosterols and phytostanols inhibit the uptake of dietary and biliary cholesterol through displacement of cholesterol. However, generally only modest reductions of serum cholesterol levels have been observed by adding free phytosterols or phytostanols to the diet.

Arteriosclerosis is a leading cause of death in many parts of the Western world. It has been shown that low-density lipoprotein (LDL) cholesterol is directly associated with the development of cardiovascular disease; whereas high-density lipoprotein (HDL) cholesterol has an inverse relationship with cardiovascular disease development.

People with combined hyperlipidemia run even higher risks of heart disease. Elevated blood serum levels of cholesterol and elevated levels of triglycerides are generally accepted both as causes and as indicators of the progression of cardiovascular disease. Thus, lowering serum cholesterol and triglyceride levels is seen as a desirable goal and a major strategy for intervention. Many methods have been proposed to lower serum cholesterol, including, for example, use of certain pharmaceutical agents and the ingestion of phytosterols in various forms. Likewise, many methods have been proposed to lower serum triglycerides, among them ingestion of polyunsaturated fatty acids (PUFAs) in various forms.

The physical properties of food additives are especially important in food applications. The physical properties of a food additive (i.e., a food ingredient) dictate the forms into which the additives may be delivered, e.g. in oils or butters. Further, certain physical properties of a food additive, for example solubility and melting point, may affect acceptability of a food product to a consumer by changing the texture, mouth feel, or taste in complicated, unpredictable ways. One problem with the use of a free phytosterol as a food additive has been its crystalline nature and limited solubility in oils. Generally, a large amount of phytosterol has been required to achieve an effect on the cholesterol level but with resultant physical problems. Thus, other forms of phytosterol have been sought.

For example, WO 96/38047 reports a fat-based food product including natural fat components that have a blood cholesterol lowering effect. This product also includes at least one of tocotrienol, oryzanol, and phytosterol with at least one component of PUFA-triglycerides. The phytosterols present in such mixtures are mainly in the free phytosterol form in low, defined concentrations, with relatively low solubility. The resultant products are semi-solids. Much higher amounts, proportionally, of the PUFA triglycerides to phytosterols are used. Effects of the mixtures on triglyceride levels are not described.

Mitchell, U.S. Pat. No. 4,588,717, discloses fatty acid esters made from a phytosterol and a C₁₈-C₂₀ fatty acid as vitamin supplements or as diet pills. Included as such fatty acids are also the unsaturated acids linolenic, linoleic, and arachidonic acid. It is generally known that these fatty acids have almost no effect on triglyceride levels in vivo.

WO 97/42830 discloses the manufacture and the use of gels consisting of partly crystallized mixtures of natural food oils with low concentrations of sterols and sterol esters (especially sitosterol and oryzanol), and optionally monoglycerides, in defined ratios to impart firmness to edible liquid fats. Because of the low sterol and sterol ester content, such products of necessity require substantial volumes of liquid and additional caloric content to deliver phytosterols and phytosterol esters in amounts to effectively lower cholesterol in vivo.

A method of reducing cholesterol in the bloodstream by administering beta-sitostanol with campestanol in defined ratios as fatty acid esters derived from vegetable oils is disclosed in WO 98/06405.

Miettinen, U.S. Pat. No. 5,502,045, also discloses the reduction of cholesterol absorption into the bloodstream by administering beta-sitostanol esters of C₂-C₂₂ acids derived from vegetable oils.

The Journal of Lipid Research, Vol. 34, pp. 1535-1544 (1998) discloses experiments wherein human subjects were fed mixtures of sitostanol esters made from rapeseed oil fatty acids. The phytostanol esters were reportedly found to reduce serum LDL cholesterol more effectively than free phytosterols, despite being hydrolyzed during intestinal passage.

The European Journal of Clinical Nutrition, Vol. 52, pp. 334-343 (1998) discloses results of human trials with margarines enriched with phytosterols and phytosterol esters. Plasma total and LDL cholesterol concentrations were shown to be reduced by sterol esters incorporated into margarine compared to controls with similar fatty acid profiles. All materials contained unsaturated fatty acid esters, especially those from oleic, linoleic, or linolenic acid. No effect was reportedly seen on plasma triglyceride concentration with these sterol-enriched margarines.

### SUMMARY OF INVENTION

Accordingly, an object of the present invention is to provide a phytosterol and/or a phytostanol ester compound produced from a reaction between a phytosterol and/or a phytostanol and a polyunsaturated fatty acid (PUFA), wherein the PUFA has from 18 to 22 carbon atoms and at least three units of unsaturation, i.e. carbon-carbon double bonds.

Another object of the invention is to provide a composition including a phytosterol and/or a phytostanol ester compound as specified above in admixture with another ester of a phytosterol and/or a phytostanol optionally also in admixture with a free phytosterol, a free phytostanol, and/or PUFA glycerides or esters. Said “another ester of a phytosterol and/or a phytostanol” is the product of the esterification reaction between a phytosterol and/or a phytostanol and a fatty acid having less than 18 or more than 22 carbon atoms and at least three carbon-carbon double bonds and/or a fatty acid having from 18 to 22 carbon atoms and less than three, including no, carbon-carbon double bonds.

A composition for lowering serum cholesterol and triglyceride levels in a mammal is a further object of the invention. This composition includes a pharmaceutically acceptable carrier in combination with an effective amount of a phytosterol and/or a phytostanol ester compound produced from a reaction between a phytosterol and/or a phytostanol and a polyunsaturated fatty acid (PUFA), wherein the PUFA has from 18 to 22 carbon atoms and at least three carbon-carbon double bonds.

A process for lowering serum cholesterol and triglyceride levels in a mammal is also another object of the invention. This process includes administering to the mammal an effective amount of a phytosterol and/or a phytostanol ester compound as defined above in combination with a pharmaceutically acceptable carrier.

Another object of the invention is a process for preparing a phytosterol and/or a phytostanol ester compound by esterification. This process includes esterifying a free phytosterol, a phytostanol or a mixture thereof with an n-3 polyunsaturated fatty acid having from 18 to 22 carbon atoms and at least three carbon-carbon double bonds.

A further object of the invention is a process for preparing a phytosterol and/or a phytostanol ester compound by interesterification. This process includes (a) mixing, in the absence of a solvent, a free phytosterol and/or a phytostanol, a fatty ester of a n-3 polyunsaturated fatty acid (PUFA), and an interesterification catalyst to form a reaction mixture; and (b) heating the reaction mixture to obtain interesterification of the phytosterol and/or phytostanol with the PUFA.

### DETAILED DESCRIPTION OF THE INVENTION

It has now been found that phytosterol and/or phytostanol esters made from the reaction of a phytosterol and/or a phytostanol with certain omega-3 polyunsaturated fatty acids (n-3 fatty acids) are surprisingly effective in reducing both serum cholesterol and triglycerides. Such polyunsaturated fatty acids include, for example, eicosapentaenoic acid (EPA) having five carbon-carbon double bonds or docosahexaenoic acid (DHA) with six carbon-carbon double bonds. These esters according to the present invention significantly lower both plasma cholesterol and triglyceride levels, while phytosterol combined with vegetable oil only lowers plasma cholesterol levels. Accordingly, the esters of the present invention may be used as a combined cholesterol reduction agent and a triglyceride-lowering agent. Thus, the compounds of the present invention positively affect two of the major risk factors for cardiovascular disease in e.g., humans.

These effects have been shown in rats, which results may be extrapolated to other mammals, such as for example, humans. The methods used and the results obtained are described in more detail below. These methods and results are illustrative only and are not intended to limit the scope of the invention in any way.

Animal treatment

Thirty male Fisher rats, weighing 177± 14 g, were maintained on a high fat diet (Table 1) during the 2 weeks preceding treatment. They were then randomly divided into five experimental groups consisting of 6 animals each. The control group (Group 1) remained on the high fat diet used during the 2-week pretreatment period. For the other experimental diets, in order to have isocaloric diets and an equal amount of fat in all the experimental diets, 2% (wt/wt) of the fat content of the control diet (1% coconut oil and 1% corn oil) was replaced by 2% (wt/wt) of the following lipids:

Group 1: Control

Group 2: 2% sitosterol mix/high oleic sunflower oil (TRISUN 80) (1:1 ratio);

Group 3: 2% sitostanol-DHA ester;

Group 4: 2% stigmasterol-EPA ester;

Group 5: 2% sitosterol mix +EPA/DHA ester (1:1 ratio)).

The fatty acid compositions of the experimental diets are shown in Table 2 below. The rats were allowed free access to water and feed, and they were maintained on a 12-hour light-dark cycle. The feed in the cages was replaced daily, all unconsumed material discarded and food intake measured. Blood samples (1 ml) were taken by retroorbital puncture at the start of the experimental period (week 0) and after 2 weeks of treatment (week 2). After 4 weeks, the animals were sacrificed by withdrawing blood from the vena cava under Isoflurane anesthesia. Blood was collected into tubes containing EDTA as an anticoagulant.

Lipid Analysis

Plasma was prepared from the heparinized blood by immediate centrifugation at 1600 g for 10 minutes at 4° C. Assays of plasma cholesterol, triglycerides, and HDL-cholesterol (precipitation method) were determined enzymatically on a COBASFARA analyzer (Roche Diagnostica, Switzerland). Non-HDL cholesterol was calculated by difference. The fatty acid composition of the diets was analyzed by gas chromatography.

Statistical Analysis

All data are expressed as means±SD (standard deviation) for animals in each diet group. The mean differences between dietary groups were analyzed by one-way analysis of covariance (ANCOVA) with subsequent Dunnet's test for multiple comparison against a control group (Group 1 and/or Group 2). The covariate adjusted for was the value of the corresponding parameter at the start of the treatment period (week 0). All tests were performed at the 5%-level and 95%-confidence intervals were calculated.

Results

The growth of rats was similar in all dietary groups during the 4-week feeding period. The average food intake for the 4-week period of the five dietary regimens was 12 g/day/rat. Dietary treatment had no significant effect on body weight and food consumption.

The plasma cholesterol was significantly lower by 28% to 46% in all the four groups treated with phytosterols relative to control and by 46% to 66% relative to the pretreatment period (week 0) (Table 3).

The HDL cholesterol levels were almost not affected by the treatment with phytosterols (Table 4). Therefore, the non-HDL cholesterol (VLDL-Cholesterol +LDL cholesterol) were mainly lowered by phytosterol treatment.

The plasma triglyceride levels were significantly lowered by 18% to 39% in the groups treated with phytosterol combined with n-3 fatty acids relative to the control group, and by 15% to 41% relative to the pretreatment period (week 0) (Table 5), whereas phytosterol combined with vegetable oil (Group 2), did not significantly lower plasma triglyceride.

TABLE 1
Composition of the rat high fat diet^{a}

| Ingredients | g/100 g anhydrous mix |
|---|---|
| Protein | 18.7 |
| Fiber | 6.6 |
| Fat | 18.3 |
| Carbohydrate | 39.2 |
| Dietary energy (MJ/Kg) | 16 |
| Metabolic energy in fat (%) | 42 |

^{a}The main fats consisted of coconut kernel (18 wt %), coconut oil (2.5 wt %), and corn oil (2.5 wt %).

The diet contained 0.5 wt % cholesterol, 1 wt % sodium cholate, and the standard vitamin and mineral mix according to the requirements for rats.

TABLE 2
Fatty acid composition of experimental diets (mol %)

| | | | | | Group 5 |
|---|---|---|---|---|---|
| | | Group 2 | Group 3 | Group 4 | 2% sitosterol |
| | Group 1 | 2% sitosterol | 2% sitostanol- | 2% stigmasterol- | mix-EPA/DHA |
| Fatty acids | Control | mix + Trisun | DHA ester | EPA ester | ester |
| Saturated | 57.73 | 56.57 | 57.62 | 56.41 | 56.86 |
| Monoenes | 18.84 | 25.35 | 15.59 | 15.62 | 16.34 |
| PUFAs | 23.43 | 18.08 | 26.79 | 27.98 | 26.81 |
| Sum n-6 | 22.08 | 16.76 | 16.85 | 16.92 | 17.47 |
| Sumn-3 | 1.21 | 1.15 | 9.91 | 10.89 | 9.20 |
| C14 | 33.91 | 33.63 | 34.99 | 34.05 | 34.04 |
| C16 | 17.84 | 16.64 | 16.76 | 16.58 | 16.66 |
| G18 | 5.38 | 5.64 | 5.33 | 5.26 | 5.42 |
| C18:1-9 | 17.99 | 24.39 | 15.08 | 15.02 | 15.16 |
| C18:1-7 | 0.55 | 0.67 | 0.41 | 0.42 | 0.56 |
| C18:2-6 | 21.91 | 16.54 | 16.31 | 16.56 | 16.74 |
| C18:3-3 | 1.21 | 1.15 | 1.17 | 1.21 | 1.25 |
| C20:5-3 | 0.00 | 0.00 | 0.11 | 9.52 | 4.56 |
| C22:6-3 | 0.00 | 0.00 | 8.58 | 0.13 | 2.76 |

Results are expressed as the percentage of fatty acid methyl esters (mol %).

TABLE 3
Effects of phytosterol esters on plasma total cholesterol in rats

| Experi- | | | | | |
|---|---|---|---|---|---|
| mental | Week 0 | Week 2 | % | Week 4 | % |
| Groups | Means ± SD | Means ± SD | change^{a} | Means ± SD | change^{a} |
| Group 1 | 2.69 ± 0.42 | 2.48 ± 0.44 | −8 | 2.24 ± 0.47^{c} | −17 |
| Group 2 | 3.25 ± 0.80 | 2.10 ± 0.31 | −35 | 1.23 ± 0.20^{b} | −62 |
| Group 3 | 2.90 ± 0.58 | 1.79 ± 0.37^{b} | −38 | 1.23 ± 0.26^{b} | −58 |
| Group 4 | 2.97 ± 0.49 | 1.94 ± 0.12^{b} | −35 | 1.61 ± 0.25^{b} | −46 |
| Group 5 | 3.58 ± 0.52 | 1.73 ± 0.26^{b} | −52 | 1.22 ± 0.21^{b} | −66 |

^{a}percent change from pretreatment.
^{b}Significantly different from control at week 2 or week 4 (P < 0.05).
^{c}Significantly different from group 2 (sitosterol mix + trisun) at week 2 or week 4 (P < 0.05).

TABLE 4
Effects of phytosterol esters on lipoproteins in rats

| | HDL Cholesterol | Non HDL cholesterol |
|---|---|---|
| | Means | Means |
| Group 1 | 0.60 ± 0.09 | 1.64 ± 0.47^{b} |
| Group 2 | 0.71 ± 0.08 | 0.52 ± 0.14^{a} |
| Group 3 | 0.49 ± 0.10^{b} | 0.75 ± 0.21^{a} |
| Group 4 | 0.53 ± 0.08 | 1.07 ± 0.26^{a,b} |
| Group 5 | 0.68 ± 0.19 | 0.54 ± 0.10^{a} |

^{a}Significantly different from control at week 2 or week 4 (P < 0.05).
^{b}Significantly different from group 2 (sitosterol mix + trisun) at week 2 or week 4 (P < 0.05).

TABLE 5
Effects of phytosterol esters on plasma triglycerides in rats

| | Week 0 | Week 2 | % | Week 4 | % |
|---|---|---|---|---|---|
| | Means ± SD | Means ± SD | change^{a} | Means ± SD | change^{a} |
| Group 1 | 1.08 ± 0.23 | 1.09 ± 0.21 | 1 | 1.22 ± 0.13 | 13 |
| Group 2 | 1.00 ± 0.17 | 1.04 ± 0.17 | 4 | 1.08 ± 0.15 | 7 |
| Group 3 | 1.25 ± 0.26 | 0.83 ± 0.13^{b} | −34 | 0.74 ± 015^{b,c} | −41 |
| Group 4 | 0.98 ± 0.15 | 0.81 ± 0.19^{b} | −17 | 0.83 ± 013^{b,c} | −15 |
| Group 5 | 1.59 ± 0.51 | 0.94 ± 0.16 | −41 | 1.00 ± 0.13^{b} | −37 |

^{a}percent change from pretreatment.
^{b}Significantly different from control at week 2 or week 4 (P < 0.05).
^{c}Significantly different from group 2 (sitosterol mix + trisun) at week 2 or week 4 (P < 0.05).

The physical properties of organic compounds, such as physical state, melting point, and solubility cannot be predicted reliably from chemical structures. As set forth above, these same properties contribute significantly to the acceptability of a food product by affecting texture, mouth feel, or taste in complex and unpredictable ways. Accordingly, the present esters of EPA and DHA were synthesized with sitosterol, sitostanol, and stigmasterol in pure form, as well as from mixtures of these and other sterols and with mixtures of these acids with other fatty acids. Some of the compounds and mixtures were liquids, whereas others were partly solid at room temperature or below. All of these compounds were significantly more soluble in edible oil than the corresponding phytosterols or phytostanols. For comparison, esters of sitostanol were synthesized with mixed fatty acids containing significant levels of C₁₆-C₂₀ unsaturated fatty acids, especially linolenic acid, as obtained from rapeseed. It was found that the mixtures produced were largely crystalline at room temperature and below. Much more food oil was required to completely dissolve these esters compared to the esters prepared with EPA or DHA.

It was further found that the compounds according to the present invention offer unique physical advantages. For example, these compounds offer a higher solubility in edible oils compared to other phytosterol esters so far described, which is advantageous for the incorporation of such compounds into a variety of food products. These materials allow co-delivery of phytosterols and/or phytostanols and selected PUFAs in their ester form in the highest concentration per unit volume possible. This is advantageous for incorporation of these materials into products where smaller volumes are important, such as in water dispersible formulations, or where additional non-essential edible oils are undesirable. The compounds of the present invention provide physical advantages over simple mixtures or formulations of other phytosterols/phytostanols and/or their fatty esters with PUFAs and their normally available ester or triglyceride forms.

The preferred phytosterols for use in the present invention are beta-sitosterol, stigmasterol, campesterol, and mixtures thereof. More preferred phytosterols are beta-sitosterol, stigmasterol, and mixtures thereof, particularly beta-sitosterol itself. The preferred phytostanols are beta-sitostanol, campestanol, and mixtures thereof. Most preferred is beta-sitostanol. Preferred PUFAs are EPA and DHA.

It is readily understood that the esters of the present invention need not be used in a pure state. Mixture of these esters may be used. Likewise, mixtures of these esters with other fatty esters of phytosterols/phytostanols may be used. The ratios of phytosterol and/or phytostanols used may vary with their source. Likewise, the ratios of PUFA and other fatty acids may vary. It is also understood that the reaction products may contain some free phytosterols/phytostanols and/or PUFA glycerides or esters. As a consequence, the physical properties of the compounds of the present invention may be varied from those with a high proportion of polyunsaturated phytosterol/phytostanol esters, which are liquids that are well soluble in edible oils, to those of a mixture with lesser proportions of unsaturation, which are semi-solid or waxy.

The compounds of the present invention may be combined with pharmaceutically acceptable carriers. In the present invention, any known carrier that is pharmaceutically acceptable and which does not interfere with the potency of the compound may be used.

When combined with a pharmaceutically acceptable carrier, the compounds of the present invention may be processed into any convenient unit dosage form. As used herein, “unit dosage form” may include for example powders, capsules, tablets, liquids, gels, and the like.

The compounds of the present invention may be administered to any mammal requiring reduction of serum cholesterol and triglycerides. In the present invention, humans are preferred examples of mammals.

A compound of the present invention may be administered to e.g., a human by any convenient process such as, for example, orally, nasally, IV, IP, anally, etc. An effective amount of a compound according to the present invention will vary based on a number of well known factors including the form of the compound used, the weight of the patient, and the route of administration. Thus, an effective amount of a composition according to the present invention may be readily determined by one skilled in the art using known dosing techniques and the data presented in the examples below.

The compounds according to the present invention may be prepared according to known methods. For example they may be obtained by esterifying a phytosterol/phytostanol with a n-3 PUFA in a known manner.

Alternatively, the compounds of the present invention may preferably be prepared by interesterification of free phytosterols and/or phytostanols with esters of n-3 PUFAs by heating in the presence of an interesterification catalyst, whereby (i) the interesterification is carried out in the absence of a solvent, (ii) the fatty esters include suitable simple C₁-C₄-esters and triglycerides, (iii) the catalyst is, for example, a sodium alkoxide of a C₁-C₄-alcohol. The reaction is suitably conducted by heating the mixture at 80-140° C. at a pressure of 133-6650 Pa whereby the reaction is preferably carried out with a stoichiometric amount to an excess of the PUFA ester.

The following examples are provided to further illustrate methods of preparation of the compounds of the present invention, as well as certain physical properties thereof. These examples are illustrative only and are not intended to limit the scope of the invention in any way.

### EXAMPLES

### Example 1

To a mixture of 0.91 g of docosahexaenoic acid (purity: 90%), 1.03 g of stigmasterol (purity: 95%) and dimethylaminopyridine (50 mg) in 18 ml of dry dichloromethane was added a solution of dicyclohexylcarbodiimide (0.63 g) in 5 ml dichloromethane. After 4 hours stirring at room temperature, the reaction was complete. Then, methanol (0.5 g) and acetic acid (0.25 g) were added and the mixture was stirred for one hour. The mixture was cooled to 0° C., filtered, and the solids rinsed with hexane (3×25 ml). The solvent was removed under reduced pressure and the residue was flash chromatographed on silica to yield a pure fraction of 1.0 g of stigmasterol docosahexaenoate as a colorless oil with consistent NMR and IR data. This substance remained in liquid form when stored for several weeks at room temperature and when cooled for several weeks at −20° C.

### Example 2

Stigmasterol eicosapenatenoate was prepared from eicosapentaenoic acid (purity: 90%) and stigmasterol using the process set forth in Example 1. Stigmasterol eicosapenatenoate (1.46 g) was obtained as a colorless oil that remained in liquid form within a temperature range of 20° C. and −20° C.

### Example 3

A mixture of eicosapentaenoic acid-docosahexaenoic acid esters of stigmasterol was prepared from stigmasterol with a mixture of 49% eicosapentaenoic acid and 27% docosahexaenoic acid using the process set forth in Example 1. The mixture of the esters of stigmasterol was obtained as a colorless oil that remained in liquid form within a temperature range of 20° C. and −20° C.

### Example 4

Stigmastanol docosahexacnoate was prepared from stigmastanol (purity: 95%) and docosahexaenoic acid (purity: 90%) using the process set forth in Example 1. Stigmastanol docosahexaenoate was obtained as a slightly colored oil that remained in liquid form between 20° C. and −20° C.

### Example 5

Stigmastanol eicosapentenoate was prepared from stigmastanol and eicosapentaenoic acid, using the process set forth in Example 1. Stigmastanol eicosapentenoate was obtained as a slightly yellowish oil that remained in liquid form within the temperature range of 20° C. and −20° C.

### Example 6

A mixture of stigmastanol eicosapentaenoic acid and docosahexaenoic acid esters was prepared from stigmastanol and a mixture of 49% eicosapentaenoic acid with 27% docosahexaenoic acid using the process set forth in Example 1. A mixture of stigmastanol eicosapentaenoic acid and docosahexaenoic acid esters was obtained as a colorless oil which became turbid when stored at 20° C. and partly solid at −20° C.

### Example 7

A mixture of sterol PUFA esters was prepared from a mixture of beta-sitosterol, campesterol, and stigmasterol and a mixture of 49% eicosapentaenoic acid with 27% docosahexaenoic acid using the process set forth in Example 1 A mixture of sterol PUFA-esters was obtained as a turbid oil containing some solids at 20° C. and partly solid at −20° C.

### Example 8

A mixture of stigmastanol unsaturated fatty esters was prepared from stigmastanol and a mixture of fatty acids obtained from basic hydrolysis of a commercial food sample of Swiss rapeseed oil (9% saturated, 61% monounsaturated, 30% polyunsaturated triglycerides) using the process set forth in Example 1. A mixture of stigmastanol unsaturated fatty esters was obtained as a colorless oil which slowly crystallized at room temperature. At −20° C. the material was essentially solid.

### Example 9

A mixture of phytosterols (20.6 g of a commercial mixture of sitosterol 43%, stigmasterol 23%, and campesterol 24% with other minor sterols) and 75% DHA-EPA ethyl esters (16.8 g of a commercial mixture of 43% ethyl docosahexaenoate and 32% ethyl eicosapentaenoate with other fatty esters) was dried at 120° C. while sparging with a stream of inert gas. To the molten mixture was added sodium ethoxide (1.03 ml 21% solution in ethanol). The mixture was stirred at 120° C. at 15 mbar vacuum for two hours. The light brown mixture was cooled to 80° C. and the catalyst quenched with dilute acid. The separated oil phase was dehydrated by heating under reduced pressure while sparging with a stream of inert gas. 35.0 g of crude phytosterol esters were obtained as a turbid light brown oil, which remained in fluid form at room temperature. HPLC showed that the conversion to sterol esters was 95%.

### Example 10

A mixture of phytosterols (148 g of a commercial mixture of sitosterol 43%, stigmasterol 23%, and campesterol 24% with other minor sterols) and fish oil glycerides (141 g of a commercial mixture of glycerides with fatty acid composition of 17% EPA and 11% DHA) was dehydrated by sparging at 120° C. with inert gas. To the molten mixture was added sodium ethoxide (11.9 ml of 21% solution in ethanol). The mixture was stirred at 120° C. at 15 mbar vacuum for one hour.

The light brown mixture was quenched with dilute acid, and the separated oil phase was dehydrated under reduced pressure to produce 249 g of a light brown oil that slowly crystallized to a semi-solid mass. HPLC showed that the conversion was 93%.

### Example 11

Solubilities of materials made according to the procedures described in Examples 1-8, as well as the parent sterols were assessed in a commercial sample of Swiss rapeseed oil by alternately adding small increments of oil at room temperature to weighed amounts of sterol esters and agitating for 5 minute periods until a solution was attained. The minimum starting ratio was about 1:1, and trials were discontinued at above 10:1.

| | Solubility g oil/ | |
|---|---|---|
| Material | g material | |
| stigmasterol docosahexaenoate | miscible | >1 |
| stigmasterol eicosapentenoate | miscible | >1 |
| stigmasterol EPA-DHA ester mixture | miscible | >1 |
| stigmastanol docosahexaenoate | miscible | >1 |
| stigmastanol eicosapentaneoate | miscible | >1 |
| stigmastanol EPA-DHA ester mixture | soluble | >4 |
| sitosterol sterols mix EPA-DHA ester mixture | miscible | >1 |
| stigmastanol rape-seed ester mixture | insoluble | >10 |
| stigmasterol | insoluble | >10 |
| stigmastanol | insoluble | >10 |
| docosahexaenoic acid ethyl ester 90% | miscible | >1 |
| EPA ethyl ester 90% | miscible | >1 |

The invention being thus described, it will be obvious that the same may be varied in many ways. Such variations are not to be regarded as a departure from the spirit and scope of the invention and all such modifications are intended to be included within the scope of the following claims.

We claim:

1. A phytosterol and/or a phytostanol ester compound produced from a reaction of a phytosterol and/or a phytostanol with a polyunsaturated fatty acid, wherein the polyunsaturated fatty acid has from 18 to 22 carbon atoms and at least three carbon-carbon double bonds.

2. A compound according to claim 1 wherein the phytosterol is selected from the group consisting of beta-sitosterol, stigmasterol, campesterol, and mixtures thereof.

3. A compound according to claim 2 wherein the phytosterol is selected from the group consisting of beta-sitosterol, stigmasterol, and mixtures thereof.

4. A compound according to claim 3 wherein the phytosterol is beta-sitosterol.

5. A compound according to claim 1 wherein the phytostanol is selected from the group consisting of campestanol, beta-sitostanol, and mixtures thereof.

6. A compound according to claim 5 wherein the phytostanol is beta-sitostanol.

7. A compound according to claim 1 wherein the polyunsaturated fatty acid is eicosapentaenoic acid or docosahexaenoic acid.

8. A composition comprising a compound according to claim 1 in admixture with another ester of a phytosterol and/or a phytostanol optionally also in admixture with a free phytosterol, a free phytostanol, and/or PUFA glycerides or esters, said another ester of a phytosterol and/or a phytostanol being the product of the esterification reaction between a phytosterol and/or a phytostanol and a fatty acid having less than 18 or more than 22 carbon atoms and at least three carbon-carbon double bonds and/or a fatty acid having from 18 to 22 carbon atoms and less than three carbon-carbon double bonds.

9. A composition for lowering serum cholesterol and triglyceride levels in a mammal comprising a pharmaceutically acceptable carrier in combination with an effective amount of phytosterol and/or a phytostanol ester compound produced from a reaction of a phytosterol and/or a phytostanol with a polyunsaturated fatty acid having from 18 to 22 carbon atoms and at least three carbon-carbon double bonds.

10. A composition according to claim 9 wherein the pharmaceutically acceptable carrier and phytosterol and/or phytostanol ester compound are formed into a unit dosage form.

11. A composition according to claim 10 wherein the unit dosage form is selected from the group consisting of capsules, powders, liquids, gels, and tablets.

12. A composition according to claim 10 wherein the composition is a dietary supplement or a food ingredient.

13. A composition according to claim 10 wherein the mammal is a human.

14. A process for lowering serum cholesterol and triglyceride levels in a mammal comprising administering to the mammal an effective amount of the compound of claim 1 in combination with a pharmaceutically acceptable carrier.

15. A process for preparing a phytosterol and/or a phytostanol ester compound comprising esterifying a free phytosterol, a phytostanol or a mixture thereof with a n-3 polyunsaturated fatty acid having from 18 to 22 carbon atoms and at least three carbon-carbon double bonds.

16. A process for preparing a phytosterol and/or a phytostanol ester compound comprising:
(a) mixing, in the absence of a solvent, a free phytosterol and/or a phytostanol, an ester of a n-3 polyunsaturated fatty acid (PUFA), and an interesterification catalyst to form a reaction mixture; and
(b) heating the reaction mixture to obtain interesterification of the phytosterol and/or the phytostanol with the ester of the n-3 PUFA.

17. A process according to claim 16 wherein the ester is a simple C₁-C₄-ester or a triglyceride.

18. A process according to claim 16 wherein the interesterification catalyst is a sodium alkoxide of a C₁-C₄-alcohol.

19. A process according to claim 16 wherein the reaction mixture is heated from about 80° C. to about 140° C. at a pressure of about 133 Pa to about 6650 Pa.

20. A process according to claim 16 wherein interestification is carried out with a stoichiometric amount to an excess of the ester of the n-3 PUFA.
